(19) United States
(12) Patent Application Publication
(10) Pub. No.: US 2016/0108107 A1
(43) Pub. Date: Apr. 21, 2016

(54) DEAMIDATED ANTI-GLUTEN ANTIBODY AND USES THEREOF

(71) Applicant: INSTITUT NATIONAL DE LA RECHERCHE AGRONOMIQUE (INRA), Paris (FR)

(72) Inventors: Sandra DENERY, Nantes (FR); Olivier TRANQUET, La Chapelle Sur Erdre (FR); Colette LARRE, Nantes (FR)

(21) Appl. No.: 14/770,464

(22) PCT Filed: Feb. 26, 2014
(86) PCT No.: PCT/IB2014/059269

§ 371 Date: Aug. 25, 2015

(30) Foreign Application Priority Data
Feb. 26, 2013 (FR) 13 51685
Apr. 9, 2013 (FR) 13 53178

(51) Int. Cl. C07K 16/16; G01N 33/68

(52) U.S. Cl. CPC C07K 16/16; G01N 33/68; C07K 2317/33; C07K 2317/565; C07K 2317/56; C07K 2317/34; G01N 2333/415

(57) ABSTRACT

The present invention relates to a monoclonal antibody that is capable of bonding to deamidated gluten proteins and has no cross-reaction with the non-deamidated gluten proteins.

### FIELD OF THE INVENTION

The present invention relates to the field of controlling the innocuousness of certain products containing deamidated gluten for human beings or animals, capable of developing allergic reactions to this type of transformed gluten.

### PRIOR ART

Gluten proteins, which encompass the gliadin and glutenin protein subunits, are constituents common in numerous food products, in particular bakery or patisserie products, and also industrial culinary preparations, or else in certain cosmetic products, it being possible for the latter to comprise protein hydrolysates and in particular wheat protein hydrolysates.

Gluten proteins are characterized in particular by their specific composition of amino acids, these proteins containing a high content of proline residues (approximately 30% of the total amino acid residues), and glutamine residues (approximately 40% of the total amino acid residues). The constituent proteins of gluten are also characterized by the presence of repeated amino acid sequences. Five classes of gliadins that are gluten constituents are known, respectively α-gliadins, β-gliadins, γ-gliadins, ω-2 gliadins and ω-5 gliadins. Two classes of glutenins are also known, respectively low-molecular-weight glutenins and high-molecular-weight glutenins.

Gluten proteins are used in industry mainly because of their viscoelastic and insolubility properties. However, the insoluble nature of gluten proteins constitutes a technical limitation to further expanding their industrial use.

Diversified use of gluten proteins has been made possible by the availability of modified glutens constituting water-dispersible or water-soluble products.

To obtain modified glutens having increased water-dispersibility or water-solubility properties, use is generally made of methods of transforming native gluten by deamidation using an acid or alkaline treatment.

However, an increasing number of cases of allergy to deamidated gluten proteins have been recorded, without an allergic reaction to the native proteins. The symptoms of allergic reactions to deamidated gluten proteins are frequently severe. These symptoms may comprise angioedema accompanied by generalized urticaria. Allergic reactions to deamidated gluten may also result in the occurrence of anaphylactic shock.

Antigenic determinants contained in deamidated gluten proteins and recognized by IgEs of allergic patients have been identified (see Denery-Papini et al., 2012, Allergy, Vol. 67: 1023-1032).

In order to prevent allergic reactions to deamidated gluten in consumers, skin tests for detecting deamidated gluten allergies have been described (see in particular Battais et al., 2006, Eur. Ann. Allergy Clin. Immunol., Vol. 38: 59-61). However, it is difficult for these skin tests to make it possible to differentiate between the existence of an allergy to wheat proteins, in particular to native gluten, and the existence of an allergy to deamidated gluten. This is because the isolated protein fractions used to carry out these skin tests contain both native proteins and deamidated proteins.

An antibody MC01 is for example known, which binds specifically to deamidated α-gliadin and does not bind to non-deamidated α-gliadin (see in particular Skovbjerg et al., 2004, Biochem. Biophys. Acta, Vol. 1690 (3): 220-230; Skovbjerg et al., 2008, Dig. Dis. Sci. Vol. 53: 2917-2924).

However, the varietal origin or the processes for transforming wheat flours that are known may nevertheless result in a final product in which the content of the various gluten proteins may substantially vary, owing in particular to the transformation methods used, such as techniques comprising steps using solvents, for example an alcohol. This is because, although gliadins are soluble in alcohol, glutenins are insoluble therein. Thus, the industrial transformation of wheat flours may result in a depletion of the flour with respect to gliadins or glutenins.

It follows that the use of antibodies specific for a given gluten protein is not suitable for the overall detection of deamidated gluten in a sample. This is because the amount of this given deamidated gluten protein compared with the other deamidated gluten proteins may be substantially reduced, which may make its detection difficult, or even impossible.

Likewise, the known processes for transforming wheat flours may result in final products comprising deamidated gluten proteins which have a very variable degree of deamidation.

There is therefore a need to provide means and processes for detecting deamidated gluten proteins, which are alternatives to or which are improved with respect to the known means and processes.

There is a need in the prior art for the development of means which make it possible to reduce or avoid the occurrence of deamidated gluten allergies in consumers.

### SUMMARY OF THE INVENTION

The present invention provides antigen-binding molecules, in particular monoclonal antibodies and antigen-binding fragments of said monoclonal antibodies, which have the property of binding specifically to at least one deamidated gluten protein, said antigen-binding molecules not exhibiting, however, any cross reaction with a non-deamidated gluten protein.

As a reminder, gluten comprises the following proteins: α-gliadins, β-gliadins, γ-gliadins, ω-2 gliadins, ω-5 gliadins, low-molecular-weight glutenins and high-molecular-weight glutenins. The low-molecular-weight glutenins have a molecular weight of less than 90 000 and the high-molecular-weight glutenins have a molecular weight greater than or equal to 90 000. Generally, gluten comprises approximately 60% to 70% by weight of gliadins and approximately 30% to 40% by weight of glutenins, relative to the total weight of the gluten. The gliadins are composed of approximately 4% to 11% by weight of ω-5 gliadins, of approximately 8% to 22% by weight of ω-2 gliadins, of approximately 26% to 29% by weight of α/β-gliadins and of approximately 10% to 26% by weight of γ-gliadins, relative to the total weight of the gluten.

The invention relates in particular to an antigen-binding molecule in the form of a monoclonal antibody having the property of binding to at least one deamidated gluten protein, said monoclonal antibodies not exhibiting any cross reaction with a non-deamidated gluten protein, said monoclonal antibody being produced by the hybridoma deposited according to the Treaty of Budapest at the CNCM on Feb. 25, 2013, under accession number 1-4717, with the Collection Nationale de Cultures de Microorganismes [National Collection of Microorganism Cultures] (CNCM) of the Institut Pasteur of Paris.

The invention also relates to an antigen-binding molecule chosen from a monoclonal antibody or an antigen-binding fragment of said monoclonal antibody, capable of binding to deamidated gluten proteins and not exhibiting any cross reaction with non-deamidated gluten proteins.

Said monoclonal antibody is in particular produced by the hybridoma deposited according to the Treaty of Budapest at the CNCM on Feb. 25, 2013, under accession number 1-4717, with the Collection Nationale de Cultures de Microorganismes [National Collection of Microorganism Cultures] (CNCM) of the Institut Pasteur of Paris.

The invention also relates to an antigen-binding molecule, in particular a monoclonal antibody, comprising at least one chain chosen from a heavy chain or a light chain of a variable region, said antigen-binding molecule comprising at least one CDR (“Complementarity Determining Region”) chosen from the CDRs which have been sequenced according to the protocol that is described in example 1 using the cells of the hybridoma deposited according to the Treaty of Budapest at the CNCM on Feb. 25, 2013, under accession number 1-4717 with the Collection Nationale de Cultures de Microorganismes [National Collection of Microorganism Cultures] (CNCM) of the Institut Pasteur of Paris.

The invention relates to an antigen-binding molecule, in particular a monoclonal antibody, comprising at least one variable region of a heavy chain (V_{H}) or one variable region of a light chain (V_{L}), said antigen-binding molecule comprising at least one CDR chosen from the following CDRs:
(a) a heavy-chain CDR chosen from the following CDRs:
   V_{H}-CDR1 comprising the sequence SEQ ID NO. 2,
   V_{H}-CDR2 comprising the sequence SEQ ID NO. 3, and
   V_{H}-CDR3 comprising the sequence SEQ ID NO. 4; or
(b) a light-chain CDR chosen from the following CDRs:
   V_{L}-CDR1 comprising the sequence SEQ ID NO. 6,
   V_{L}-CDR2 comprising the sequence SEQ ID NO. 7, and
   V_{L}-CDR3 comprising the sequence SEQ ID NO. 8.

According to a first aspect, the antigen-binding molecules according to the invention are in particular characterized in that they bind specifically to a deamidated gluten protein chosen from a deamidated gliadin and a deamidated glutenin and do not exhibit any cross reaction with a non-deamidated gliadin or a non-deamidated glutenin.

According to a second aspect, the antigen-binding molecules according to the invention are in particular characterized in that they bind to deamidated gliadins chosen from the group comprising deamidated α-gliadins, deamidated β-gliadins, deamidated γ-gliadins, deamidated ω-2 gliadins and deamidated ω-5 gliadins and do not exhibit any cross reaction with the corresponding non-deamidated gliadins.

According to a third aspect, the antigen-binding molecules according to the invention are in particular characterized in that they bind to deamidated low-molecular-weight glutenins and do not exhibit any cross reaction with non-deamidated low-molecular-weight glutenins.

According to another aspect, the antigen-binding molecules according to the invention are in particular characterized in that they bind to deamidated high-molecular-weight glutenins and do not exhibit any cross reaction with non-deamidated high-molecular-weight glutenins.

According to another aspect, the antigen-binding molecules according to the invention are in particular characterized in that they are capable of binding to a deamidated gliadin and a deamidated glutenin and do not exhibit any cross reaction with a non-deamidated gliadin and a non-deamidated glutenin.

The invention also relates to a complex formed between an antigen-binding molecule as defined above and a deamidated gluten protein.

The present invention also relates to processes and kits for detecting the presence of a deamidated gluten protein in a sample, in particular of deamidated gluten proteins, for which an antigen-binding molecule according to the invention is used.

### DESCRIPTION OF THE FIGURES

FIG. 1 illustrates the capacity of a monoclonal antibody according to the invention to bind to a variety of deamidated glutens without exhibiting any cross reaction with a non-deamidated gluten protein (ω-5 gliadin). Along the x-axis: increasing dilutions of the monoclonal antibody. Along the y-axis: absorbence value (OD) at 490 nanometers. Curves: “♦”: deamidated gliadin; “▪”: deamidated gluten No. 1; “▴”: deamidated gluten No. 2; “x”: deamidated gluten No. 3; “*”: non-deamidated native ω-5 gliadin.

FIG. 2 illustrates the capacity of a monoclonal antibody according to the invention to bind to constituent deamidated proteins of deamidated gluten, without exhibiting any cross reaction with native gluten proteins. Along the x-axis: increasing dilutions of the monoclonal antibody. Along the y-axis: absorbence value (OD) at 490 nanometers. Curves: No. 1 “-♦- α”: native α-gliadin; No. 2 “-▴-γ”: native γ-gliadin; No. 6 “-*-ω-5”: native ω-5 gliadin; No. 7 “ [symbol] -LMW”: native low-molecular-weight glutenin; No. 9 “- [symbol] -α-D”: deamidated α-gliadin; No. 11 “▪-ω2-D”: deamidated ω-2 gliadin; No. 12 “x-ω5-D”: deamidated ω-5 gliadin; No. 5 “▪-β”: native β-gliadin; No. 8 “x-ω2”: native ω-2 gliadin; No. 4 “ HMW”: native high-molecular-weight glutenin; No. 3 “ [symbol] BSA”: native bovine serum albumin; No. 13 “♦ γ-D”: deamidated γ-gliadin; No. 10 “▴ Glut. LMW-D”: deamidated low-molecular weight glutenin.

FIG. 3 illustrates the capacity of a monoclonal antibody according to the invention to bind to deamidated gliadins without exhibiting any cross reaction with native gliadins, evaluated according to an immunological assay of the competitive ELISA type. Along the x-axis: percentage inhibition of the binding of the monoclonal antibody to the LQPEEPFPEQC peptide (SEQ ID NO. 22). Curves: “▴”: native gliadins; “♦”: deamidated gliadins.

FIG. 4 illustrates the absence of specificity of a monoclonal antibody according to the invention with respect to industrial flours such as wheat flour, rye flour, barley flour, oat flour, hard wheat flour, spelt flour, corn flour and soybean flour. Along the x-axis, dilutions to 1/10 (dil 10) and to 1/20 (dil 20) of the flours tested. Along the y-axis: percentage inhibition of the competitive ELISA Assay by the flour tested.

### DETAILED DESCRIPTION OF THE INVENTION

In order to make available means for reducing or avoiding the occurrence of deamidated gluten allergies in consumers, the applicant has developed preventive means, in the form of processes and kits which for the first time allow a reliable and reproducible detection of the presence of deamidated gluten proteins in a sample, for instance a food product or a cosmetic product.

The processes and kits provided by the present invention may also be used to comply with the possible legal or regulatory requirements relating to measuring the presence of gluten in food products. It is in particular recalled that the Codex Alimentarius established by the World Health Organization makes it obligatory to inform consumers about the presence or absence of gluten in foods (standard ALINORM 08/31/26).

For example, the Skeritt antibody, which is directed against wheat gliadin and which recognizes high-molecular-weight glutenins and w-gliadin, is known.

The R5 antibody, which is directed against rye secalin, and has good affinity for wheat gliadin, and also certain soybean and lupin proteins, is also known.

However, to the knowledge of the applicant, the gluten detection tests currently available do not make it possible to effectively detect the presence of deamidated gluten (Kanerva et al., 2011, J. Cereal Sci. 53, 335-339).

It is specified that the availability of the processes and kits for detecting deamidated gluten proteins which are specified in the present description has been made possible because the applicant has obtained antigen-binding molecules, and in particular monoclonal antibodies, capable of binding selectively to deamidated gluten proteins.

### Antigen-Binding Molecule

The present invention firstly provides antigen-binding molecules which bind to at least one deamidated gluten protein and do not exhibit any cross reaction with a non-deamidated gluten protein.

In particular, the present invention provides an antigen-binding molecule chosen from a monoclonal antibody and an antigen-binding fragment of said monoclonal antibody, capable of binding to deamidated gluten proteins and not exhibiting any cross reaction with non-deamidated gluten proteins.

For the purposes of the present description, a binding molecule capable of binding to deamidated gluten proteins encompasses a binding molecule capable of binding to any deamidated gluten protein.

The present invention provides an antigen-binding molecule chosen from a monoclonal antibody and an antigen-binding fragment of said monoclonal antibody, capable of binding to a deamidated gliadin and a deamidated glutenin and not exhibiting any cross reaction with a non-deamidated gliadin and a non-deamidated glutenin.

In certain embodiments, an antigen-binding molecule chosen from a monoclonal antibody and an antigen-binding fragment of said monoclonal antibody, according to the present invention, is capable of binding to a deamidated gliadin, i.e. a deamidated α-gliadin, deamidated β-gliadin, deamidated γ-gliadin, deamidated ω-2 gliadin and deamidated ω-5 gliadin, and does not exhibit any cross reaction with a non-deamidated gliadin.

In certain embodiments, an antigen-binding molecule chosen from a monoclonal antibody and an antigen-binding fragment of said monoclonal antibody according to the present invention, is capable of binding to a deamidated glutenin, i.e. a deamidated high-molecular-weight glutenin and a deamidated low-molecular-weight glutenin and does not exhibit any cross reaction with a non-deamidated glutenin.

In particular, the invention provides an antigen-binding molecule in the form of a monoclonal antibody characterized in that it is produced by the hybridoma deposited according to the Treaty of Budapest at the CNCM on Feb. 25, 2013, under accession number 1-4717, with the Collection Nationale de Cultures de Microorganismes [National Collection of Microorganism Cultures] (CNCM) of the Institut Pasteur of Paris.

According to the invention, the term “antigen-binding molecule” is intended to mean a protein capable of binding selectively to at least one deamidated gluten protein, and not exhibiting any cross reaction with a non-deamidated gluten protein. According to the invention, the term “antigen-binding molecule” is intended to mean more particularly a protein capable of binding selectively to deamidated gluten proteins, and not exhibiting any cross reaction with non-deamidated gluten proteins. The antigen-binding molecules encompass essentially, if not exclusively, monoclonal antibodies and antigen-binding fragments originating from monoclonal antibodies.

According to the invention, the term “monoclonal antibody” is intended to mean an antibody originating from a virtually homogeneous or totally homogeneous population of antibodies. More specifically, in a population of monoclonal antibodies, the individual antibodies are identical, with the exception of naturally occurring mutations that may be found in very small proportions. Thus, for the purposes of the invention, a “monoclonal antibody” encompasses a single molecule of an antibody, and equally a homogeneous population of monoclonal antibodies, originating from the growth of a single cell clone, such as a hybridoma, or else a eukaryotic host cell transfected or transformed with a nucleic acid encoding said antibody.

According to the invention, the term “antigen-binding fragment” or “functional fragment” of an antibody is intended to mean a part of a parent antibody comprising a region involved in the binding of the parent antibody to the target antigen, i.e. to a target epitope of the antigen of interest. An “antigen-binding fragment” or “functional fragment” encompasses a fragment chosen from an Fv fragment (variable fragment), an scFv fragment (single-chain variable fragment), a Fab fragment, a F(ab′)₂ fragment, a Fab′ fragment, an Fabc fragment, an scFv-Fc fragment, “diabodies” and also any other antibody fragment which has retained the antigen-binding ability of the parent antibody from which it derives, in the present case the ability to bind selectively to at least one deamidated gluten protein, even more particularly the ability to bind selectively to deamidated gluten proteins.

According to the invention, the antigen-binding ability of a monoclonal antibody or of a functional fragment thereof, in the case in point its ability to bind to at least one deamidated gluten protein, in particular to bind to deamidated gluten proteins, may be verified by carrying out an ELISA assay, as described later in the description, and even more specifically in the examples.

According to the invention, an antigen-binding molecule is characterized in that it does not exhibit any cross reaction with a non-deamidated gluten protein. The absence of cross reaction with a non-deamidated gluten protein is, for the purposes of the present invention, determined as indicated hereinafter.

It is possible to carry out an ELISA immunological assay comprising the following steps:
a) providing a support on which a gluten protein to be tested is immobilized,
b) incubating the support provided in step a) with an antigen-binding molecule as defined in the present description, under conditions which allow the formation of complexes between said gluten protein and said antigen-binding molecule, and
c) measuring a signal representative of the amount of antigen-binding molecules bound to said gluten protein.

In one particular variant of the ELISA immunological assay above, step c) comprises the following steps:
c1) incubating the support obtained at the end of step b) with a ligand which recognizes the antigen-binding molecule, preferably an antibody which recognizes the antigen-binding molecule, said ligand being labeled with a detectable molecule, and
c2) measuring the signal generated by the detectable molecule, said signal being representative of the amount of antigen-binding molecules bound to said gluten protein.

In certain embodiments of the ELISA immunological assay above, the detectable molecule is an enzyme, for instance peroxidase, and step c2) is the following:
c2) incubating the support obtained at the end of step c1) with a substrate of said enzyme, for example with a peroxidase substrate, and measuring the signal generated by the product resulting from the conversion of the substrate, preferentially by measuring the light absorbence value at the wavelength of absorption of light by said product.

A specific embodiment of the ELISA immunological assay of the above type is illustrated in the examples.

The immunological assay is carried out with reference deamidated gliadins which may be derived from a total gliadin fraction or chosen from deamidated α-gliadins and deamidated β-gliadins or deamidated γ-gliadins. Entirely preferably, the reference deamidated gliadins are deamidated γ-gliadins.

The percentage cross reaction of the antigen-binding molecule with a non-deamidated gluten protein is then determined according to formula (I) below:

% CROSS=value of the “Protein X” signal/value of the “GLUT-ND” signal (I),

in which:
% CROSS is the cross reaction value,
“Protein X” is chosen from non-deamidated α-gliadin, non-deamidated β-gliadin and non-deamidated high-molecular-weight glutenin,
“GLUT-ND” is the deamidated gluten protein used as reference, and
the value of the signal is the value of the measurement of the signal generated by the binding of the antigen-binding protein to the deamidated or non-deamidated gluten protein under consideration, for example the value of absorbence of light by an enzymatic conversion product at a given wavelength.

The antigen-binding molecule is considered not to exhibit any cross reaction with a non-deamidated gluten protein when the “% CROSS” value is less than or equal to 10, and preferentially less than or equal to 5.

According to the invention, an “antigen-binding fragment” or “functional fragment” also encompasses a protein comprising at least one of the CDRs (“Complementarity Determining Regions”), i.e. one of the regions of the antibody which determines the complementarity of the antibody with the antigen, which are located in the hypervariable regions of the variable region of the parent antibody.

For the purposes of the invention, a “CDR” is the region of an immunoglobulin which determines the antigen-binding (“Complementarity Determining Region”) and signifies a hypervariable region of immunoglobulin light chains and heavy chains, as defined by Kabat (Kabat et al., Sequences of proteins of immunological interest, 5th Ed., US Department of Health and Human Services, NIH, 1991). An immunoglobulin comprises three heavy-chain CDRs and three light-chain CDRs. For the purposes of the present description, the terms “CDR” and “CDRs” mean, as appropriate, one, several or all the regions containing most of the amino acid residues which are responsible for the ability of an antibody to bind selectively to a target antigen, i.e. to an epitope of said target antigen. According to another definition, “CDR” or “CDRs” means the hypervariable regions of immunoglobulin heavy and light chains, as defined according to the IMGT numbering. As a reminder, IMGT unique numbering was defined for the purposes of comparing immunoglobulin variable domains, independently of the type of antigen receptor, of the type of chain or of the identity of the species from which the immunoglobulins originate.

The present invention relates in particular to an antigen-binding molecule comprising at least one CDR which has a sequence having at least 80% amino acid identity with a CDR chosen from the CDRs of sequences SEQ ID NOs. 2, 3, 4, 6, 7 and 8, as defined according to the IMGT numbering system.

These CDRs have been sequenced in accordance with the protocol described in example 1, using the hybridoma deposited according to the Treaty of Budapest at the CNCM on Feb. 25, 2013, under accession number 1-4717, with the Collection Nationale de Cultures de Microorganismes [National Collection of Microorganism Cultures] (CNCM) of the Institut Pasteur of Paris.

For the purposes of the present invention, the “percentage identity” between two amino acid sequences or of nucleic acid sequences is determined by comparing the two optimally aligned sequences, through a comparison window.

The part of the nucleotide sequence in the comparison window may thus comprise additions or deletions (for example “gaps”) compared with the reference sequence (which does not comprise these additions or these deletions) so as to obtain an optimal alignment between the two sequences.

The percentage identity is calculated by determining the number of positions at which an identical amino acid (or an identical nucleic base) is observed for the two sequences compared, then by dividing the number of positions at which there is identity between the two amino acids (or between the two nucleic bases) by the total number of positions in the comparison window, then by multiplying the result by one hundred in order to obtain the percentage amino acid (or nucleotide) identity of the two sequences with respect to one another.

The optimal alignment of the sequences for the comparison may be carried out on a computer by means of known algorithms.

Entirely preferably, the percentage sequence identity is determined by means of the CLUSTAL W software (version 1.82), the parameters being set as follows: (1) CPU MODE=ClustalW mp; (2) ALIGNMENT=“full”; (3) OUTPUT FORMAT=“aln w/numbers”; (4) OUTPUT ORDER=“aligned”; (5) COLOR ALIGNMENT=“no”; (6) KTUP (word size)=“default”; (7) WINDOW LENGTH=“default”; (8) SCORE TYPE=“percent”; (9) TOPDIAG=“default”; (10) PAIRGAP=“default”; (11) PHYLOGENETIC TREE/TREE TYPE=“none”; (12) MATRIX=“default”; (13) GAP OPEN=“default”; (14) END GAPS=“default”; (15) GAP EXTENSION=“default”; (16) GAP DISTANCES=“default”; (17) TREE TYPE=“cladogram” and (18) TREE GAP DISTANCES=“hide”.

For the purposes of the invention, an amino acid sequence having at least 80% amino acid identity with a reference amino acid sequence encompasses the amino acid sequences having at least 81%, 82%, 83%, 84%, 85%, 86%, 87%, 88%, 89%, 90%, 91%, 92%, 93%, 94%, 95%, 96%, 97%, 98% or at least 99% amino acid identity with said reference sequence.

For the purposes of the invention, a nucleotide sequence having at least 80% nucleotide identity with a reference nucleotide sequence encompasses the nucleotide sequences having at least 81%, 82%, 83%, 84%, 85%, 86%, 87%, 88%, 89%, 90%, 91%, 92%, 93%, 94%, 95%, 96%, 97%, 98% or at least 99% nucleotide identity with said reference sequence.

The present invention relates to an antigen-binding molecule, in particular a monoclonal antibody or an antigen-binding fragment originating from a monoclonal antibody, comprising at least one variable region of a heavy chain comprising at least one CDR chosen from the following CDRs:
V_{H}-CDR1, comprising a sequence having at least 80% amino acid identity with the sequence SEQ ID NO. 2,
V_{H}-CDR2, comprising a sequence having at least 80% amino acid identity with the sequence SEQ ID NO. 3, and
V_{H}-CDR3, comprising a sequence having at least 80% amino acid identity with the sequence SEQ ID NO. 4.

The present invention relates to an antigen-binding molecule, in particular a monoclonal antibody or an antigen-binding fragment originating from a monoclonal antibody, comprising at least one variable region of a heavy chain comprising at least one CDR chosen from the following CDRs:
V_{H}-CDR1, comprising the sequence SEQ ID NO. 2,
V_{H}-CDR2, comprising the sequence SEQ ID NO. 3, and
V_{H}-CDR3, comprising the sequence SEQ ID NO. 4.

The present invention also relates to an antigen-binding molecule, in particular a monoclonal antibody or an antigen-binding fragment originating from a monoclonal antibody, comprising at least one variable region of a heavy chain comprising the following CDRs:
V_{H}-CDR1, comprising a sequence having at least 80% amino acid identity with the sequence SEQ ID NO. 2,
V_{H}-CDR2, comprising a sequence having at least 80% amino acid identity with the sequence SEQ ID NO. 3, and
V_{H}-CDR3, comprising a sequence having at least 80% amino acid identity with the sequence SEQ ID NO. 4.

The present invention also relates to an antigen-binding molecule, in particular a monoclonal antibody or an antigen-binding fragment originating from a monoclonal antibody, comprising at least one variable region of a heavy chain comprising the following CDRs:
V_{H}-CDR1, comprising the sequence SEQ ID NO. 2,
V_{H}-CDR2, comprising the sequence SEQ ID NO. 3, and
V_{H}-CDR3, comprising the sequence SEQ ID NO. 4.

The present invention relates to an antigen-binding molecule, in particular a monoclonal antibody or an antigen-binding fragment originating from a monoclonal antibody, comprising at least one variable region of a light chain comprising at least one CDR chosen from the following CDRs:
V_{L}-CDR1, comprising a sequence having at least 80% amino acid identity with the sequence SEQ ID NO. 6,
V_{L}-CDR2, comprising a sequence having at least 80% amino acid identity with the sequence SEQ ID NO. 7, and
V_{L}-CDR3, comprising a sequence having at least 80% amino acid identity with the sequence SEQ ID NO. 8.

The present invention relates to an antigen-binding molecule, in particular a monoclonal antibody or an antigen-binding fragment originating from a monoclonal antibody, comprising at least one variable region of a light chain comprising at least one CDR chosen from the following CDRs:
V_{L}-CDR1, comprising the sequence SEQ ID NO. 6,
V_{L}-CDR2, comprising the sequence SEQ ID NO. 7, and
V_{L}-CDR13, comprising the sequence SEQ ID NO. 8.

The invention also relates to an antigen-binding molecule, in particular a monoclonal antibody or an antigen-binding fragment originating from a monoclonal antibody, comprising at least one variable region of a light chain comprising the following CDRs:
V_{L}-CDR1, comprising a sequence having at least 80% amino acid identity with the sequence SEQ ID N6,
V_{L}-CDR2, comprising a sequence having at least 80% amino acid identity with the sequence SEQ ID NO. 7, and
V_{L}-CDR3, comprising a sequence having at least 80% amino acid identity with the sequence SEQ ID NO. 8.

The invention also relates to an antigen-binding molecule, in particular a monoclonal antibody or an antigen-binding fragment originating from a monoclonal antibody, comprising at least one variable region of a light chain comprising the following CDRs:
V_{L}-CDR1, comprising the sequence SEQ ID N6,
V_{L}-CDR2, comprising the sequence SEQ ID NO. 7, and
V_{L}-CDR3, comprising the sequence SEQ ID NO. 8.

The present invention also relates to an antigen-binding molecule, in particular a monoclonal antibody, comprising:
(a) at least the variable region of a heavy chain comprising the following three CDRs:
   V_{H}-CDR1, comprising a sequence having at least 80% amino acid identity with the sequence SEQ ID NO. 2,
   V_{H}-CDR2, comprising a sequence having at least 80% amino acid identity with the sequence SEQ ID NO. 3, and
   V_{H}-CDR3, comprising a sequence having at least 80% amino acid identity with the sequence SEQ ID NO. 4, and
(b) at least the variable region of a light chain comprising the following three CDRs:
   V_{L}-CDR1, comprising a sequence having at least 80% amino acid identity with the sequence SEQ ID NO. 6,
   V_{L}-CDR2, comprising a sequence having at least 80% amino acid identity with the sequence SEQ ID NO. 7, and
   V_{L}-CDR3, comprising a sequence having at least 80% amino acid identity with the sequence SEQ ID NO. 8.

The present invention also relates to an antigen-binding molecule, in particular a monoclonal antibody, comprising:
(a) at least the variable region of a heavy chain comprising the following three CDRs:
   V_{H}-CDR1, comprising the sequence SEQ ID NO. 2,
   V_{H}-CDR2, comprising the sequence SEQ ID NO. 3, and
   V_{H}-CDR3, comprising the sequence SEQ ID NO. 4, and
(b) at least the variable region of a light chain comprising the following three CDRs:
   V_{L}-CDR1, comprising the sequence SEQ ID NO. 6,
   V_{L}-CDR2, comprising the sequence SEQ ID NO. 7, and
   V_{L}-CDR3, comprising the sequence SEQ ID NO. 8.

An antibody suitable for the present invention may be selected from monoclonal antibodies from mouse, rat, goat, rabbit, horse, llama, human and other primate.

In certain embodiments, the antigen-binding molecule consists of a murine monoclonal antibody, preferably of IgG1 isotype.

Preferentially, said murine monoclonal antibody consists of the monoclonal antibody produced by the hybridoma deposited at the CNCM on Feb. 25, 2013, under accession number 1-4717, or of a functional fragment thereof.

The invention also relates to an antigen-binding molecule, in particular a monoclonal antibody, comprising at least one heavy chain comprising a variable region, said variable region having a sequence having at least 80% amino acid identity with the sequence SEQ ID NO. 1.

The invention also relates to an antigen-binding molecule, in particular a monoclonal antibody, comprising at least one heavy chain comprising a variable region, said variable region having a sequence comprising the sequence SEQ ID NO. 1.

The invention also relates to an antigen-binding molecule, in particular a monoclonal antibody, comprising at least one light chain comprising a variable region, said variable region having a sequence having at least 80% amino acid identity with the sequence SEQ ID NO. 5.

The invention also relates to an antigen-binding molecule, in particular a monoclonal antibody, comprising at least one light chain comprising a variable region, said variable region having a sequence comprising the sequence SEQ ID NO. 5.

The invention also relates to an antigen-binding molecule, which encompasses a monoclonal antibody, comprising:
at least the variable region of a heavy chain having a sequence comprising at least 80% amino acid identity with the sequence SEQ ID NO. 1, and
at least the variable region of a light chain having a sequence comprising at least 80% amino acid identity with the sequence SEQ ID NO. 5.

The invention also relates to an antigen-binding molecule, i.e. a monoclonal antibody, comprising:
at least the variable region of a heavy chain comprising the sequence SEQ ID NO. 1, and
at least the variable region of a light chain comprising the sequence SEQ ID NO. 5.

In certain embodiments, the antigen-binding molecule comprises a variable heavy chain (V_{H}) and a variable light chain (V_{L}) bonded together by a linker peptide. The linker peptide may comprise from 1 to 20 amino acids in length, which encompasses 1, 2, 3, 4, 5, 6, 7, 8, 9, 10, 11, 12, 13, 14, 15, 16, 17, 18, 19 or 20 amino acids in length. The linker peptide preferentially consists of glycine and/or serine amino acids. An illustrative example of a linker peptide is the Gly₄Ser peptide. The linker peptide may be a multimer of the Gly₄Ser monomer motif, for example (Gly₄Ser)2, (Gly₄Ser)₃ or else (Gly₄Ser)₄.

The antigen-binding molecules of the scFv type may be prepared for example as described by McCafferty et al. (1990, Nature, Vol. 348; 552-554) or else in PCT application No. WO 92/01047.

In certain embodiments, the antigen-binding molecule comprises a peptide structure on to which is grafted at least one CDR, said CDR being grafted in such a way as to preserve all or some of the properties of recognition of the target antigen by the paratope, as in the parent antibody from which said CDR originates.

Preferably, in these embodiments of an antigen-binding molecule according to the invention, one or more of the sequences of CDRs defined in the present description are present within the peptide structure, so as to reconstitute a peptide backbone which promotes folding of the grafted CDRs, allowing preservation of the properties of recognition of the target antigen by the paratope, as in the parent antibody from which said CDR originates.

As peptide structures that are of use for the grafting of CDRs, mention may in particular be made of fibronectin, preferentially fibronectin type III domain 10, lipocalin, anticalin (see Skerra et al., 2001, J. Biotechnol, Vol. 74(4): 257-275), protein Z originating from the B domain of Staphylococcus aureus protein A, thioredoxin A or else proteins comprising repeat motifs, such as ankyrin repeat protein (see Kohl et al., PNAS, 2003, Vol. 100 (4): 1700-1705), repeat sequences of the “armadillo repeat” type, found for example in β-catenin, leucine-rich repeat sequences found for example in tropomyosin or tropomodulin, or repeat sequences of the “tetratricopeptide repeat” type found for example in p67phox subunit of NADPH oxidase. Mention may also be made of the peptide structures derived from toxins or else protein inhibitors of neuronal NO synthase (PIN). An illustration of the use of PIN as a peptide structure for the grafting of CDRs is described by Bes et al. (2006, Biochem. Biophys. Res. Commun., Vol. 343(1): 334-344). Mention may also be made of the grafting of CDRs on to one of the loops of neocarzinostatin, as described by Nicaize et al. (2004, Protein Science, Vol. 13(7): 1882-1891).

An antigen-binding molecule according to the invention encompasses chimeric antibodies, and where appropriate also humanized antibodies.

Chimeric antibodies are antibodies containing a natural variable region (heavy chain and light chain) originating from an antibody of a first given species, in combination with the constant regions of the light chain and of the heavy chain originating from an antibody of a second species, different than the first species.

The antigen-binding molecules according to the invention, in particular the antibodies according to the invention, may be prepared by using genetic recombination techniques. For example, a chimeric antibody may be prepared by cloning a DNA comprising a promoter and a sequence encoding the variable region of a non-human monoclonal antibody of the invention, including of a murine monoclonal antibody of the invention, and the sequence encoding the constant region of another antibody, for example the constant region of another murine antibody or else the constant region of another human antibody. Such a chimeric antibody according to the invention may be, for example, a mouse-mouse chimeric antibody or a mouse-human chimeric antibody, the specificity for a deamidated gluten protein being determined by the variable region of said chimeric antibody and the isotype being determined by the constant region of said chimeric antibody. Chimeric or humanized antibodies may be prepared according to the techniques described by Jones et al. (1986, Nature, Vol. 321: 522-525), by Verhoeyen etal. (1988, Science, Vol. 239: 1534-1536) or else by Riechmann etal. (1988, Nature, Vol. 322: 323-327). Chimeric or humanized antibodies may also be prepared according to techniques known to those skilled in the art, such as those described by Singer et al. (1992, J. Immun., Vol. 150: 2844-2857), Mountain et al. (1992, Biotechnol. Genet. Eng. Rev., Vol. 10: 1-142) or else Bebbington et al. (1992, Biotechnology, Vol. 10: 169-175). Other techniques for preparing antibodies by genetic recombination that may be implemented according to the invention, which includes CDR grafting techniques, are for example those described in the following patent documents: EP 0 451 216, EP 0 682 040, EP 0 939 127, EP 0 566 647, U.S. Pat. No. 5,530,101, U.S. Pat. No. 6,054,297, U.S. Pat. No. 5,886,152 or else U.S. Pat. No. 5,877,293.

The present invention also relates to a nucleic acid encoding an antigen-binding molecule as defined in the present description, and in particular a monoclonal antibody as defined in the present description, or encoding a functional fragment thereof.

In particular, the invention relates to a nucleic acid encoding a polypeptide comprising a CDR of an antigen-binding molecule defined in the present description, said CDR being chosen from the CDRs which have a sequence having at least 80% amino acid identity with the CDRs sequenced in accordance with the protocol of example 1.

The present invention also relates to a nucleic acid chosen from:
a nucleic acid having at least 80% identity with the polynucleotide of sequence SEQ ID NO. 10 encoding the V_{H}-CDR1 of sequence SEQ ID NO. 2,
a nucleic acid having at least 80% identity with the polynucleotide of sequence SEQ ID NO. 11 encoding the V_{H}-CDR2 of sequence SEQ ID NO. 3,
a nucleic acid having at least 80% identity with the polynucleotide of sequence SEQ ID NO. 12 encoding the V_{H}-CDR3 of sequence SEQ ID NO. 4,
a nucleic acid having at least 80% identity with the polynucleotide of sequence SEQ ID NO. 14 encoding the V_{L}-CDR1 of sequence SEQ ID NO. 6,
a nucleic acid having at least 80% identity with the polynucleotide of sequence SEQ ID NO. 15 encoding the V_{L}-CDR2 of sequence SEQ ID NO. 7,
a nucleic acid having at least 80% identity with the polynucleotide of sequence SEQ ID NO. 16 encoding the V_{L}-CDR3 of sequence SEQ ID NO. 8.
   The present invention also relates to a nucleic acid chosen from:
a nucleic acid having at least 80% identity with the polynucleotide of sequence SEQ ID NO. 9 encoding a heavy chain of sequence SEQ ID NO. 1,
a nucleic acid having at least 80% identity with the polynucleotide of sequence SEQ ID NO. 13 encoding a light chain of sequence SEQ ID NO. 5.
   The present invention relates to a nucleic acid chosen from:
a nucleic acid comprising the polynucleotide of sequence SEQ ID NO. 10 encoding the V_{H}-CDR1 of sequence SEQ ID NO. 2,
a nucleic acid comprising the polynucleotide of sequence SEQ ID NO. 11 encoding the V_{H}-CDR2 of sequence SEQ ID NO. 3,
a nucleic acid comprising the polynucleotide of sequence SEQ ID NO. 12 encoding the V_{H}-CDR3 of sequence SEQ ID NO. 4,
a nucleic acid comprising the polynucleotide of sequence SEQ ID NO. 14 encoding the V_{L}-CDR1 of sequence SEQ ID NO. 6,
a nucleic acid comprising the polynucleotide of sequence SEQ ID NO. 15 encoding the V_{L}-CDR2 of sequence SEQ ID NO. 7,
a nucleic acid comprising the polynucleotide of sequence SEQ ID NO. 16 encoding the V_{L}-CDR3 of sequence SEQ ID NO. 8.

The present invention also relates to a nucleic acid chosen from:
a nucleic acid comprising the polynucleotide of sequence SEQ ID NO. 9 encoding the variable region of a heavy chain of sequence SEQ ID NO. 1,
a nucleic acid comprising the polynucleotide of sequence SEQ ID NO. 13 encoding the variable region of a light chain of sequence SEQ ID NO. 5.

For the purposes of the invention, the terms “nucleic acid”, “nucleic sequence” and “polynucleotide” may be used interchangeably to denote a nucleotide sequence, which may comprise modified nucleotides, and which may be a single-stranded DNA, a double-stranded DNA or a product of transcription of these DNAs.

The invention also relates to vectors comprising a nucleic acid as specified in the present description.

The vectors preferably comprise elements which enable the expression of the nucleic acids of the invention in a host cell. The vectors comprise a promoter, transcription initiation and termination signals and also appropriate transcription regulation sequences. Mention may, for example, be made of the expression vectors and systems described in patent document EP 0 380 068.

The invention also relates to a process for producing an antigen-binding molecule as defined in the present description, in particular for producing a monoclonal antibody as defined in the present description, said process comprising the following steps:
a) culturing host cells capable of producing an antigen-binding molecule,
b) recovering the antigen-binding molecules produced by said host cells.

These techniques are well known to those skilled in the art. By way of illustration, the host cells capable of producing said antigen-binding molecule encompass the cells of the hybridoma deposited according to the Treaty of Budapest at the CNCM on Feb. 25, 2013, under accession number 1-4717, with the Collection Nationale de Cultures de Microorganismes [National Collection of Microorganism Cultures] (CNCM) of the Institut Pasteur of Paris.

The invention also relates to cloning vectors and to expression vectors into which is inserted a nucleic acid encoding an antigen-binding molecule as defined in the present description, or a functional fragment of said antigen-binding molecule.

The invention also relates to host cells, for example E. coli cells, which have been transfected with an expression vector as defined above.

### Process for Preparing an Antigen-Binding Molecule in Accordance with the Present Invention

An antigen-binding molecule according to the invention, and in particular a monoclonal antibody according to the invention, may be obtained by immunization of a mammal with an immunogenic compound comprising an appropriate peptide.

Those skilled in the art are aware of numerous methods for immunizing a mammal for the purpose of the production of antibodies directed against an antigenic peptide of interest.

In certain embodiments, a non-human mammal, for example a rodent such as a mouse, a rat, a guinea pig or a rabbit, is immunized with an immunogenic composition comprising an immunogenic compound capable of inducing the production of antibodies directed against a peptide of interest.

In certain embodiments, the immunogenic compound comprises the antigenic peptide of interest which is covalently bonded to a carrier protein such as KLH (Keyhole Limpet Hemocyanin), which may be prepared according to techniques well known to those skilled in the art.

In the immunogenic composition, the immunogenic compound may be combined with one or more immunoadjuvant substance(s), such as complete Freund's adjuvant, incomplete Freund's adjuvant, aluminum hydroxide, or else an adjuvant presented in the form of an emulsion, for instance SEPPIC Immunoadjuvant substances well known to those skilled in the art are described, for example, by Petrovsky et al. (2004, Immunology and Cell Biology, Vol. 82: 488-496) or F. Vogel (1995, A compendium of adjuvants and excipients, Pharm. Biotechnol., Vol. 6: 141-228).

In one preferred embodiment, the antigenic peptide of interest comprises the sequence QPEEPFPE (SEQ ID NO. 20), which may also be denoted “epitope sequence”.

This epitope is naturally carried by γ-gliadin and by ω-2 gliadins, but not by β-gliadin and ω-5 gliadins, nor by high-molecular-weight or low-molecular-weight glutenins.

In another preferred embodiment, an antigenic peptide of interest that is particularly suitable comprises the sequence LQPEEPFPEQC (SEQ ID NO. 26).

In another particularly preferred embodiment, an antigenic peptide of interest that is suitable for obtaining an antigen-binding molecule according to the invention, and in particular a monoclonal antibody according to the invention, consists of the sequence LQPEEPFPEQC (SEQ ID NO. 26).

Surprisingly, the inventors have been able to obtain an antigen-binding molecule according to the invention, and in particular a monoclonal antibody, capable of binding to deamidated gluten proteins and not exhibiting any cross reaction with non-deamidated gluten proteins, by means of an immunization protocol which is shorter than the protocol conventionally routinely used. In the conventional protocol, at least 3 separate immunizations are carried out 3 weeks apart, and the lymphocytes producing the antibodies are extracted from the spleen of an immunized mouse.

In a particularly preferred protocol, 17 days separate the first immunization and the cell fusion aimed at immortalizing the lymphocytes secreting the antibodies of interest.

Contrary to the conventional protocol, the mice are immunized in the feet and the lymphocytes are extracted from the popliteal lymph nodes. The brevity of the immunization protocol and the fact that the lymphocytes are taken from secondary lymph nodes rather than from the spleen (primary lymphatic organ), after 2 to 3 months of immunization, make it possible to obtain antibodies covering a broader reactivity range.

In the case in point, such a protocol is entirely suitable for producing an antigen-binding molecule according to the invention, and in particular a monoclonal antibody, capable of binding to deamidated gluten proteins and not exhibiting any cross reaction with non-deaminated gluten proteins.

### Detection Processes and Kits

The applicant has shown that an antigen-binding molecule according to the invention, and in particular a monoclonal antibody according to the invention, has the following properties:

said antigen-binding molecule, in particular said monoclonal antibody, binds to a deamidated gluten protein, and is capable of binding to deamidated gluten proteins, i.e. to deamidated α-gliadins, to deaminated β-gliadins, to deamidated γ-gliadins, to deamidated ω-2 gliadins, to deamidated ω-5 gliadins, to deamidated high-molecular-weight glutenins and to deamidated low-molecular-weight glutenins,
said antigen-binding molecule, in particular said monoclonal antibody, does not exhibit any cross reaction, for the purposes of the invention, with an non-deamidated gluten protein,
said antigen-binding molecule, in particular said monoclonal antibody, binds with high affinity to a deamidated gluten protein, i.e. complexes between an antigen-binding molecule according to the invention and a deamidated gluten protein may be detected using a reduced amount of said antigen-binding molecule, in particular a reduced amount of a monoclonal antibody of the invention.

The above advantageous properties of an antigen-binding molecule according to the invention, in particular of a monoclonal antibody according to the invention, have allowed the applicant to design processes for detecting the presence, in a test sample, of deamidated gluten, or of deamidated gluten proteins. More particularly, the advantageous specificity and affinity properties of an antigen-binding molecule according to the invention, including a monoclonal antibody according to the invention, have allowed the applicant to develop processes for detecting the presence of deamidated gluten proteins which have specificity, sensitivity and reproducibility characteristics that are compatible with industrial use, in particular for the purposes of detecting deamidated gluten proteins in commercial products intended to come into contact with the human or animal body, including products for food use and cosmetic products.

Consequently, the present invention also relates to the use of an antigen-binding molecule as defined in the present description, in particular a monoclonal antibody as defined in the present description, for detecting, in vitro, the presence of a deamidated gluten protein in a sample, more particularly the presence of deamidated gluten proteins.

It also relates to an antigen-binding molecule as defined in the present description, for use thereof in an in vitro method for detecting the presence of deamidated gluten in a sample, more particularly in the presence of deamidated gluten proteins.

The invention also relates to a process for detecting the presence of a deamidated gluten protein, in particular of deamidated gluten proteins, in a sample, comprising the following steps:
a) providing a test sample,
b) bringing said sample into contact with an antigen-binding molecule as defined in the present description,
c) detecting the complexes possibly formed between said sample and said antigen-binding molecule.

In step a), it is possible to use any type of sample for which the presence of at least one deamidated gluten protein is sought, including final food or cosmetic products or else glutens used as raw materials for producing final products. In certain embodiments, the sample consists of an aqueous suspension obtained from the crude product to be analyzed. In other embodiments, the sample consists of an aqueous solution obtained from the crude product to be analyzed. In yet other embodiments, the sample consists of a fraction of the crude product to be analyzed, it being possible for said fraction to be prepared using any technique which makes it possible to separate several constituents of the crude product to be analyzed and thus to enrich the sample with proteins, or even to selectively enrich the sample with gluten or with certain constituent proteins of gluten. There are numerous such separation techniques and they are part of the general knowledge of those skilled in the art.

The high sensitivity of the above process is possible because of the good affinity of the antigen-binding molecule for deamidated gluten proteins. It is why the above process may be carried out using a sample of final product in which the gluten content is reduced.

In certain embodiments of the above process, the sample consists of a sample of gluten which may be used as an ingredient in the production of a large variety of products, including food products and cosmetic products.

As has already been indicated, an antigen-binding molecule as defined in the present description may be advantageously used for detecting the presence of deamidated gluten in a sample, more particularly of deamidated gluten proteins, for legal or regulatory purposes, in particular in order to comply with the current and future requirements of the Codex Alimentarius established by the World Health Organization.

The implementation of steps b) and c) is part of the general knowledge of those skilled in the art.

Step c) may also be defined as a step consisting in detecting the binding or the attachment of said antigen-binding molecule to said sample.

The attachment of the antigen-binding molecule to the test sample may be detected according to a variety of known techniques, which includes in particular assays of ELISA or RIA type or else immunoblotting techniques (also called Western blotting).

In certain embodiments, the above process consists of an immunological assay according to the ELISA technique well known to those skilled in the art.

By way of illustration, the support on which the antibody is immobilized may be a porous or nonporous, water-insoluble material. The support may be hydrophilic or capable of being made hydrophilic, and comprises inorganic powders such as silica, magnesium aluminum sulfate; natural polymer materials, in particular cellulose-based materials and cellulose-derived materials; natural or synthetic polymers such as nitrocellulose, cellulose acetate, poly(vinyl chloride), polyacrylamide, crosslinked dextran, agarose, polyacrylate, polyethylene, polypropylene, poly(4-methylbutene), polystyrene, polymethacrylate, poly(ethylene terephthalate), nylon, poly(vinyl butyrate), certain types of glass such as Bioglass, or ceramics.

The attachment of an antibody according to the invention to a support may be carried out by techniques well known to those skilled in the art. The support may be in diverse forms, including in the form of strips or of particles such as beads. The surface of the support may be polyfunctional or capable of being polyfunctionalized so as to attach the antibody via covalent or noncovalent interactions which may be specific or nonspecific.

By way of illustration, for the immobilization of antibodies on a support, those skilled in the art may advantageously refer to U.S. Pat. No. 4,168,146 or to U.S. Pat. No. 4,347,311.

In step c), the detection of the complexes formed between the sample and the antigen-binding molecule, i.e. the detection of the attachment of the antigen-binding molecule to the sample, may be carried out by measuring a signal generated by a detectable molecule bonded to said antigen-binding molecule. As is known, the detectable molecule may be directly bonded, for example by covalent bonding, to the antigen-binding molecule, or else the detectable molecule is bonded to a ligand which attaches noncovalently to the antigen-binding molecule. For example, said ligand may be of the type such as an antibody labeled with a detectable molecule, said antibody recognizing a region of the antigen-binding molecule other than the antigen-binding region. Typically, in step c), a labeled antibody which recognizes a region of the antigen-binding molecule that is not involved in recognition of the antigen is used. In the embodiments of the process in which the antigen-binding molecule is a monoclonal antibody, a ligand which binds selectively to the constant part of said monoclonal antibody is preferentially used. In these embodiments, the ligand is preferentially an antibody, commonly called “secondary antibody”, which binds to the constant part of the anti-deamidated gluten monoclonal antibody.

Preferably, in the embodiment in which one of the antibodies is immobilized on a support, the other antibody is covalently bonded to a molecule which allows it to be directly or indirectly detected.

The detectable molecule may be isotopic or nonisotopic.

By way of illustration, but which is nonlimiting, the detectable molecule may be involved in a catalytic reaction, such as an enzyme, an enzyme fragment, an enzyme substrate, an enzyme inhibitor, a coenzyme or a catalyst. The detectable molecule may also be a chromogen, such as a fluorophor, a dye or a chemiluminescent molecule.

The detectable molecule may thus be a fluorescent molecule, such as the molecules described by Ichinose et al. (1991, Fluorometric Analysis in Biomedical Chemistry. New York: Wiley-Interscience) or else fluorescent isothiocyanate derivatives, phycoerythrin, rhodamine isothiocyanate, dansyl chloride or else the XRITC compound, GFP (Green Fluorescent Protein) of the fish Aequorea victoria and its numerous derivatives, or else YFP (Yellow Fluorescent Protein), and also the luciferase protein.

Among the detectable molecules which have a catalytic activity, the preferred molecules are the following enzymes, according to the I.U.B. International Classification: (i) class 1 oxidoreductases and (ii) class 3 hydrolases. The preferred oxidoreductases are (i) dehydrogenases of class 1.1, more particularly 1.1.1, 1.1.3 and 1.1.99, and (ii) peroxidases of class 1.11 and (iii) hydrolases of class 3.1, and more particularly of class 3.1.3 and of class 3.2, more particularly 3.2.1. The preferred dehydrogenases are malate dehydrogenase, glucose-6-phosphate dehydrogenase and lactate dehydrogenase. The preferred oxidase is glucose oxidase. The preferred peroxidase is horse radish peroxidase. The preferred hydrolases are alkaline phosphatases, ss-glucosidase and lyzozyme.

The detectable molecule may also be a radioactively labeled molecule, for example labeled with an isotope chosen from [³H], [³²P] and [¹²⁵I].

In the embodiment in which the detectable molecule constitutes an indirect label, one of the constituent antibodies of a detection kit according to the invention may be covalently bonded to a ligand such as biotin or to streptavidin.

In this particular embodiment, the detectable molecule is chosen in such a way that it attaches to the ligand covalently bonded to the antibody. The detectable molecule may, for example, itself be bonded respectively to biotin or streptavidin.

According to yet another embodiment of a detection kit according to the invention, the means for revealing the formation of a complex between a deamidated gluten protein present in the sample tested and an antigen-binding molecule as defined in the present description may be an antibody, for example, in the embodiments in which the antigen-binding molecule is a monoclonal antibody, an antibody capable of binding specifically to the Fc part of the monoclonal antibody or else an antibody capable of attaching specifically to the isotype to which the anti-deamidated gluten protein monoclonal antibody belongs.

The detectable molecules encompass enzymes such as peroxidase, alkaline phosphatase, α-D-galactosidase, glucose oxidase, glucose amylase, carbonic anhydrase, acetylcholine esterase, lysozyme, malate dehydrogenase, or else glucose-6-phosphate dehydrogenase. The detectable molecules also encompass biotin, digoxigenin and 5-bromodeoxyuridine. The detectable molecules also encompass fluorescent molecules such as fluorescein and derivatives thereof, GFP (“Green Fluorescent Protein”), YFP (“Yellow Fluorescent Protein”), or else umbelliferone. The detectable molecules also encompass chemiluminescent molecules such as luminol and dioxetanen, and bioluminescent molecules such as luciferase and luciferin. The detectable molecules also encompass radioactive labels such as iodine¹²³, iodine¹²⁵, iodine¹²⁶, iodine¹³³, bromine⁷⁷, technetium^{99m}, indium¹¹¹, gallium⁶⁷, gallium⁶⁸, rubenium⁹⁵, rubenium⁹⁷, rubenium¹⁰³, rubenium¹⁰⁵, mercuryl¹⁰⁷, mercury²⁰³, rhenium^{99m}, rhenium¹⁰¹, rhenium¹⁰⁵, scandium⁴⁷, fluorine¹⁸ and iodine¹³¹.

The methods for labeling compounds with detectable molecules are well known to those skilled in the art. By way of illustration, for the labeling of compounds with radioactive molecules, those skilled in the art may refer to the article by Hunter et al. (1962, Nature, Vol. 194: 495), or else to patent documents U.S. Pat. No. 4,424,200 and U.S. Pat. No. 4,479,930.

Generally, for carrying out immunological detection assays, those skilled in the art may advantageously refer to the following book: “Immunoassay”, Eds Eleftherios P Diamandis and Theodore K Christopoulos, Academic Press, 1996. In particular, for carrying out antibody labeling techniques, those skilled in the art may refer to chapter 6 of the abovementioned book “Immunoassay”.

By way of illustration, the examples describe an embodiment of the above detection process in which:
step b) is carried out by incubating an anti-deamidated gluten monoclonal antibody (antigen-binding molecule according to the invention) in wells of a plate in which proteins originating from the test sample, for example the constituent gluten of the test sample, have been previously immobilized,
step c) is carried out by incubating the wells previously subjected to step b) with a labeled antibody which binds to the constant part of the heavy chain of the anti-deamidated gluten monoclonal antibody, in the case in point a peroxidase-labeled anti- IgG1 antibody.

In certain embodiments of the above detection process, said process consists of a “sandwich”-type immunological assay and comprises the following steps:
a) providing a solid support on which is immobilized an antigen-binding molecule as defined in the present description, for example an anti-deamidated gluten monoclonal antibody,
b) incubating said solid support with, or bringing the latter into contact with, a test sample, under conditions suitable for the attachment of deamidated gluten proteins to said binding molecule previously immobilized on said support,
c) incubating the solid support obtained at the end of step b) with, or bringing the latter into contact with, an antigen-binding molecule as defined in the present description, for example an anti-deamidated gluten monoclonal antibody, and
d) detecting the possible presence of deamidated gluten proteins bound to said antigen-binding molecule.

The present invention also relates to “competitive” immunological assay processes well known to those skilled in the art, the principle of which is based on competition for binding to the antibody between (i) the deamidated gluten protein molecules that may be present in the sample to be analyzed and (ii) decoy molecules, for example peptides which mimic deamidated gluten proteins. In the competitive immunological assays, the presence of deamidated gluten proteins induces an inhibition of the binding of the anti-deamidated gluten antibody to the decoy peptides, the level of inhibition of the binding of the antibody to the decoy peptides correlating with the amount of deamidated gluten proteins which is present in the test sample.

Thus, the invention also relates to a process for detecting the presence of a deamidated gluten protein, more particularly of deamidated gluten proteins, in a sample, comprising the following steps:
a) providing a test sample,
b) incubating said sample with an antigen-binding molecule as defined in the present description, in particular an anti-deamidated gluten monoclonal antibody, under conditions which allow the formation of complexes between said antigen-binding molecule and a deamidated gluten protein, in particular deamidated gluten proteins, in order to obtain an incubation solution,
c) bringing the incubation solution obtained at the end of step b) into contact with a support on which is immobilized a peptide which mimics a deamidated gluten protein, which may also be called “decoy peptide”, and
d) detecting or quantifying the complexes possibly formed between (i) said peptide which mimics a deamidated gluten protein and which is immobilized on the support and (ii) said antigen-binding molecule present in the incubation solution obtained at the end of step b).

Advantageously, in step d) of the above process, a signal generated by the formation of the complexes between (i) said peptide which mimics a deamidated gluten protein and which is immobilized on the support, and (ii) said antigen-binding molecule present in the incubation solution obtained at the end of step b), is measured. The value of said signal correlates with the amount of said complexes that are formed at the end of step c). Thus, the attachment of antigen-binding molecules to the decoy peptides may be quantified in step d) of the process. The amount of antigen-binding molecules which are attached to the decoy peptides at the end of step d) may be expressed in arbitrary units representative of the level of attachment, for example in light absorbence value (assay of ELISA type) or in radioactivity value (RIA assay).

Advantageously, the above process comprises an additional step (step e)) of comparing the quantitative value obtained at the end of step d). The reference value is preferentially obtained by carrying out the above process by providing, in step a), a sample free of deamidated gluten protein, which encompasses carrying out the above process by providing, in step a), a sample containing at least one non-deamidated gluten protein, said sample not containing any deamidated gluten protein.

The ratio between (i) the quantification value measured in step d) when the process is carried out with the test sample and (ii) the reference value makes it possible to calculate the percentage inhibition of the attachment of the antigen-binding molecule to the decoy peptide. The higher the concentration of deamidated gluten proteins in the test sample, the higher the percentage inhibition of the attachment of the antigen-binding molecule to the decoy peptide.

In certain embodiments of the process, the above process is carried out beforehand successively with a series of reference samples each containing a known concentration of deamidated gluten proteins, in order to generate a standard curve of the percentage inhibitions. In these embodiments, the amount of deamidated gluten proteins present in the test sample is determined on the standard curve, on the basis of the quantitative value obtained at the end of step d) of the process.

In certain embodiments of the above detection process, the peptide which mimics a deamidated gluten protein comprises an amino acid sequence chosen from:

(SEQ ID NO. 17)
EPEEPFPQ,
(SEQ ID NO. 18)
EPQEPFPE,
(SEQ ID NO. 19)
EPEQPFPE,
(SEQ ID NO. 20)
QPEEPFPE,
and
(SEQ ID NO. 21)
EPEEPFPE.

In certain embodiments of the above detection process, the peptide which mimics a deamidated gluten protein comprises an amino acid sequence LQPEEPFPEQC (SEQ ID NO. 22).

The invention also relates to a kit for detecting the presence of deamidated gluten in a sample, comprising at least one antigen-binding molecule as defined in the present description, and, where appropriate, a reagent or a plurality of reagents required for carrying out a detection process as defined in the present description.

Features relating to a detection kit according to the invention have already been previously described in relation to the processes for detecting the presence of deamidated gluten proteins.

In certain embodiments of the detection kit, the antigen-binding molecule, which may be an anti-deamidated gluten monoclonal antibody as defined in the present description, is immobilized on a support, for example is immobilized in wells of plates of an appropriate type for carrying out immunological assays, for example in wells of microplates appropriate for carrying out ELISA or RIA immunological assays, well known to those skilled in the art.

The present invention therefore provides an antigen-binding molecule dedicated to detecting the presence of deamidated gluten proteins in a sample originating from a product intended for human or veterinary food.

The present invention also provides an antigen-binding molecule dedicated to detecting the presence of a plurality of deamidated gluten proteins present in a sample originating from a product intended for human or veterinary food.

The present invention also provides an antigen-binding molecule dedicated to detecting the presence of a plurality of deamidated gluten proteins, which may have variable degrees of deamidation, present in a sample originating from a product intended for human or veterinary food.

The present invention therefore provides an antigen-binding molecule dedicated to detecting the presence of a very low amount of a plurality of deamidated gluten proteins, of different nature, present in a sample originating from a product intended for human or veterinary food.

The present invention is also illustrated, without in any way being limited thereto, by the examples which follow.

### EXAMPLES

### Example 1

### Production of Monoclonal Antibodies According to the Invention

3 mice are immunized in the hind feet by injection of 30 μl of a mixture (v/v) of a solution of peptide of sequence SEQ ID NO. 20 (QPEEPFPE) conjugated to KLH and of a solution of adjuvant of the brand name TitermaxGold. 17 days later, a booster was given under the same conditions. Finally, 3 days later, the mice are put to death and their popliteal lymph nodes are removed. The cells present in the lymph nodes are extracted by perfusion and fused with myeloma cells (NS 1 line).

The hybridomas are then selected according to conventional antibody production processes.

### Example 2

### Characterization of the Monoclonal Antibody PEE 14C7

### 2.1. Isotyping

The monoclonal antibody produced by the PEE 14C7 hybridoma (hybridoma deposited according to the Treaty of Budapest at the CNCM on Feb. 25, 2013, under accession number 1-4717, with the Collection Nationale de Cultures de Microorganismes [National Collection of Microorganism Cultures] (CNCM) of the Institut Pasteur of Paris) was subjected to isotyping using the kit sold by the company Pierce under the reference No. 26178, according to the manufacturer's recommendations.

It was determined that the monoclonal antibody produced by the PEE 14C7 hybridoma is of IgG1 isotype.

### 2.2. Sequencing

The monoclonal antibody produced by the PEE 14C7 hybridoma was subject to sequencing by the company GenScript (Piscataway, United States). In summary, a step of extraction of the total RNA from the hybridoma cells which had been previously frozen was carried out. The cDNA was synthesized by amplification of the regions encoding the antibody, and in particular by amplification of the nucleic acids encoding the variable regions of the heavy chains and of the light chains, by the RT-PCR technique using the total RNA. The amplified cDNAs encoding respectively the variable regions of the heavy chains and of the light chains of the monoclonal antibody were cloned into separate vectors. Then, after screening of the positive colonies, the DNA was sequenced. The amino acid and nucleotide sequences corresponding to the heavy and light chains, and also the CDRs, are respectively indicated in Tables 1 and 2 below.

TABLE 1
Amino acid sequences of the heavy and light
chains and of the corresponding CDRs

| Reference | Nature of the sequence | SEQ ID NO. |
|---|---|---|
| V_{H} | Heavy chain variable region | 1 |
| V_{H}-CDR1 | Heavy chain, CDR1 | 2 |
| V_{H}-CDR2 | Heavy chain, CDR2 | 3 |
| V_{H}-CDR3 | Heavy chain, CDR3 | 4 |
| V_{L} | Light chain variable region | 5 |
| V_{L}-CDR1 | Light chain, CDR1 | 6 |
| V_{L}-CDR2 | Light chain, CDR2 | 7 |
| V_{L}-CDR3 | Light chain, CDR3 | 8 |

TABLE 2
Nucleotide sequences of the heavy and light
chains and of the corresponding CDRs

| Reference | Nature of the sequence | SEQ ID NO. |
|---|---|---|
| V_{H} | Heavy chain | 9 |
| V_{H}-CDR1 | Heavy chain, CDR1 | 10 |
| V_{H}-CDR2 | Heavy chain, CDR2 | 11 |
| V_{H}-CDR3 | Heavy chain, CDR3 | 12 |
| V_{L} | Light chain | 13 |
| V_{L}-CDR1 | Light chain, CDR1 | 14 |
| V_{L}-CDR2 | Light chain, CDR2 | 15 |
| V_{L}-CDR3 | Light chain, CDR3 | 16 |

### Example 3

### Specificity of a Detection Process Using an Antigen-Binding Molecule

### 3.1. Non-competitive ELISA Immunological Assay Protocol

100 μl of proteins at 1 μg/ml in 50 mM carbonate buffer, pH 9.6, are incubated overnight at 4° C. at the bottom of a 96-well, bottom, plate (Nunc, Maxisorb). After 3 washes with PBS-0.05% Tween 20, an incubation with PBS-2% (w/v) milk is carried out. The PEE 14C7 monoclonal antibody (100 μl of various dilutions of antibody in PBS-0.1% (w/v) milk) is incubated for 1 h at ambient temperature. After washes with PBS-0.05% Tween 20, 100 μl of the peroxidase-coupled goat anti-mouse IgG secondary antibody (ref: 170-6516, Bio-Rad) diluted to 1/3000 is incubated for 1 h at ambient temperature under the same conditions as the primary antibody. After 3 washes, the OPD (0-phenylenediamine, ref: P-1526, Sigma) substrate is incubated for 30 min in the dark. The reaction is stopped by adding 50 μl of H2SO₄ 4N. The optical densities are read at dual wavelength (490 nm for measuring the hydrolysis of the substrate and 630 nm for eliminating the background noise) on a plate spectrometer (Bioteck, E1x808-1).

3.2. Specificity with Respect to the Various Deamidated Gluten Proteins

The specificity of the PEE 14C7 antibody was controlled by indirect ELISA by depositing, on the solid phase, various proteins purified from gluten. These proteins were optionally subjected to chemical deamidation (0.1 N HCl; 90° C.; 1 h). The degrees of deamidation of these products were determined using the method described by Khun et al. (1996) and Gourbeyre et al. (2011). The results are given in Table 3 below.

TABLE 3

| | | | | | Low- | |
|---|---|---|---|---|---|---|
| | Alpha/ | | Omega- | Omega- | molecular- | |
| Deamidated | Beta- | Gamma- | 2 | 5 | weight | Total |
| proteins | gliadins | gliadins | gliadins | gliadins | glutenins | gliadins |
| Degree of | 52% | 32% | 36% | 51% | ND | 53% |
| deamidation | | | | | | |

Briefly, the proteins are dissolved at 1 mg/ml in 50% ethanol (native proteins) or in carbonate (0.05 M); 0.1% SDS, before being diluted to 1 μg/ml in 0.05 M carbonate for depositing in a microplate (overnight at 4° C.). All the wells of the microplate are then saturated with PBS-0.1% milk (1 h at ambient temperature). The culture supernatant of the PEE 14C7 clone is then serially diluted and deposited in the wells of the microplate. After washing in PBS-0.05% Tween 20, the monoclonal antibodies which have specifically bound to the proteins deposited in the wells are revealed with a secondary antibody (Goat anti-Mouse IgG; Biorad ref. 170-6516) conjugated to peroxidase and revealed with ortho-phenylenediamine and reading of the absorbencies at 490 nm.

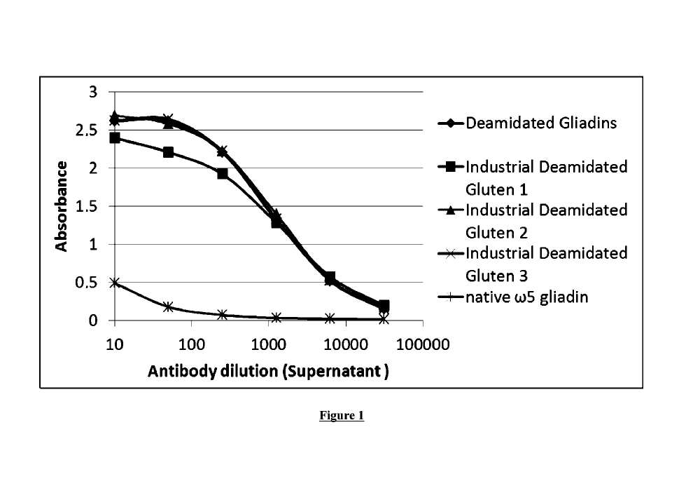

The results are given in FIG. 1. The results of FIG. 1 show that the PEE 14C7 monoclonal antibody selectively recognizes the deamidated gluten proteins, respectively deamidated α-gliadin, deamidated γ-gliadin, deamidated gliadin, deamidated ω-5 gliadin and deamidated low-molecular-weight glutenin.

3.3. The PEE 14C7 Antibody Does Not Exhibit any Cross Reaction with Non-Deamidated gluten

The percentages of reactivity of the antibody with respect to each protein are determined as described in Tranquet et al. (2012). In the present case, the percentages are determined at the 1/50 dilution of the culture supernatant (i.e. the strongest dilution which makes it possible to obtain the strongest absorbence on the best-recognized protein—deamidated gamma-gliadins). The percentage recognition of a protein X is obtained by calculating the Protein X absorbence/deamidated gamma-gliadin absorbence ratio. The results are given in Table 4 below.

TABLE 4

| | | | | | | | | Deamidated gluten proteins | | | | |
|---|---|---|---|---|---|---|---|---|---|---|---|---|
| | Native gluten proteins | | | | | | | deam. | deam. | deam. | deam. | deam. |
| | α | β | γ | ω2 | ω5 | HMW | LMW | α | γ | ω2 | ω5 | LMW |
| PEE 14C7 | 1% | 4% | 1% | 39% | 7% | 2% | 26% | 76% | 100% | 94% | 99% | 97% |
| antibody | | | | | | | | | | | | |

2.4. Specificity with Respect to Various Commercial Deamidated Glutens

The specificity of the PEE 14C7 monoclonal antibody and of the detection process which uses this monoclonal antibody was tested with respect to a variety of commercial deamidated glutens. A non-deamidated commercial comparative gluten was also used.

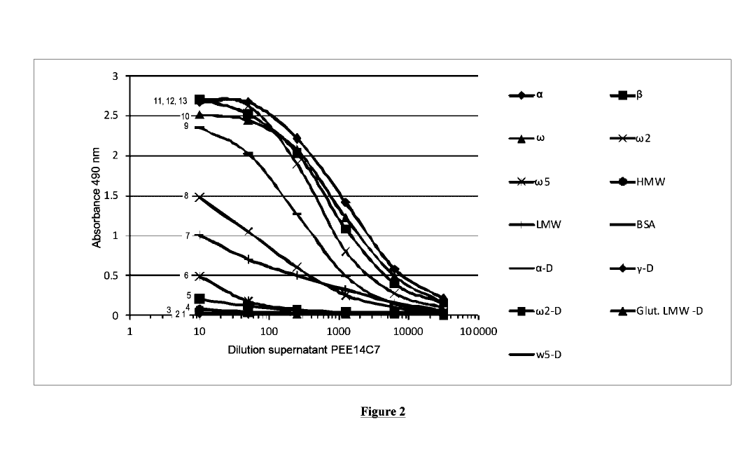

The results are represented in FIG. 2. The results of FIG. 2 show that the PEE 14C7 monoclonal antibody selectively recognizes the deamidated glutens.

### Example 4

### Sensitivity of a Detection Process Using an Antigen-Binding Molecule

It is advantageous to use the “Competitive ELISA” assay technique.

This assay based on the use of the LQPEEPFPEQC peptide (SEQ ID NO. 22) conjugated to BSA (100 ng/well) and of the PEE 14C7 antibody supernatant diluted to 1/4000 makes it possible to specifically detect deamidated gliadins at a concentration of between 1 and 8 ng/ml.

### 4.1. Competitive ELISA Protocol:

The culture supernatant of the PEE 14C7 clone, diluted to 1/4000 in PBS-0.1% milk, is preincubated (1 h 30 at ambient temperature) with, as competitor, a range of native or deamidated gliadins diluted, in PBS-0.1% milk, to various concentrations (1.6 ng/ml to 5 μg/ml), then deposited in a microplate previously coated with BSA-LQPEEPFPEQC SEQ ID NO. 22 (100 ng/well—overnight) and saturated with PBS-4% milk (1 h at ambient temperature). The washes, the saturation and the detection of the immune complexes are carried out with the same protocol as the indirect ELISA. The percentage inhibition is calculated according to the formula: [1−((OD Competitor−OD supernatant background noise)/(OD without positive Competitor−OD serum background noise))×100].

### 4.2. Results

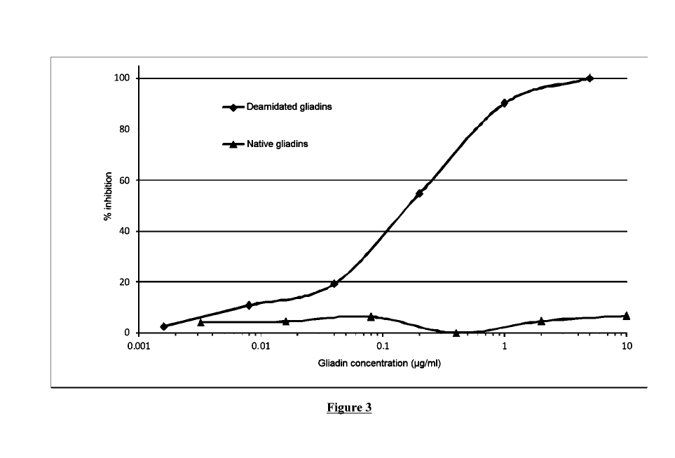

The results are given in FIG. 3. The results of FIG. 3 show the high sensitivity and the high specificity of a process for detecting the presence of deamidated gluten proteins which uses the PEE 14C7 antibody.

TABLE 5
Sequences of the peptides which mimic
a deamidated gluten protein (decoy)

| SEQ ID NO. | Type | Description |
|---|---|---|
| 17 | Peptide | Decoy-1 |
| 18 | Peptide | Decoy-2 |
| 19 | Peptide | Decoy-3 |
| 20 | Peptide | Decoy-4 |
| 21 | Peptide | Decoy-5 |
| 22 | Peptide | Decoy-competitive ELISA |

### Example 5

### Sensitivity of the Detection of the Deamidated Gluten Proteins

3 deamidated gluten sample solutions at 1000, 200 and 40 ng/ml, diluted in PBS (Phosphate Buffered Saline)-0.1% skimmed milk, and a standard range of deamidated gluten (1.6 ng/ml to 5 μg/ml) are assayed in triplicate in the competitive ELISA protocol described in paragraph 4.1. A calibration curve correlating the concentration of the standard and the percentage inhibition is established and the effective concentration of the samples prepared at 1000, 200 and 40 ng/ml is deduced therefrom. This experiment is carried out 3 times using the same deamidated gluten sample solutions at 1000, 200 and 40 ng/ml.

TABLE 6

| Amount of | | Amount of | | Coefficient | Coefficient |
|---|---|---|---|---|---|
| deamidated | | deamidated | | of intra- | of inter- |
| gluten added | | gluten measured | Degree of | assay | assay |
| to the sample | | in the sample | recovery | variation | variation |
| (ng/ml) | Assay | (ng/ml) | (%) | (%) | (%) |
| 1000 | 1 | 882 ± 54 | 88 | 6.1 | 7.8 |
| | 2 | 1045 ± 31 | 105 | 3.0 | |
| | 3 | 1049 ± 62 | 105 | 5.9 | |
| 200 | 1 | 202 ± 12 | 101 | 6.2 | 5.1 |
| | 2 | 222 ± 5 | 111 | 2.1 | |
| | 3 | 203 ± 5 | 101 | 2.7 | |
| 40 | 1 | 50 ± 3 | 124 | 5.5 | 20.3 |
| | 2 | 61 ± 4 | 152 | 6.1 | |
| | 3 | 64 ± 3 | 134 | 4.4 | |

### Example 6

### Absence of Cross Reaction with Other Cereals

100 mg of wheat flour, rye flour, barley flour, oat flour, hard wheat flour, spelt flour, corn flour and soybean flour are suspended in 1 ml of PBS (Phosphate Buffered Saline) for 1 h at ambient temperature. After centrifugation (10 min; 2500×g), the supernatant of each extraction is collected. These supernatants are then diluted to 1/10 (dil 10) and 1/20 (dil 20) in PBS-0.1% milk, and then assayed according to the competitive ELISA described in paragraph 4.1.

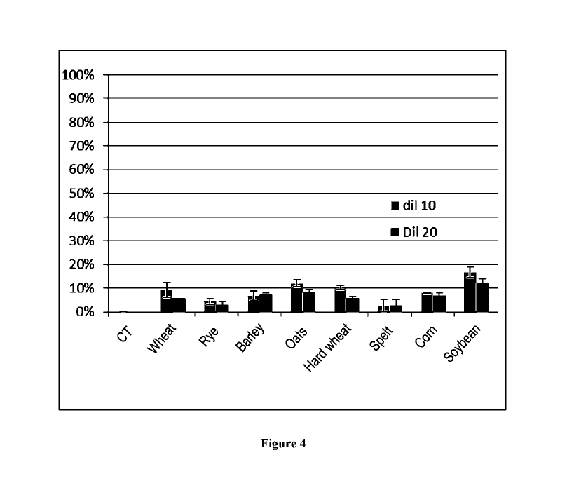

FIG. 4 shows that, for a dilution to 1/20 of an extract of the test flour, the percentage inhibition of the competitive ELISA assay by this extract remains below 15%, i.e. below the significant threshold.

1. An antigen-binding molecule chosen from a monoclonal antibody and an antigen-binding fragment of said monoclonal antibody, capable of binding to deamidated gluten proteins and not exhibiting any cross reaction with a non-deamidated gluten protein.

2. An antigen-binding molecule chosen from a monoclonal antibody and an antigen-binding fragment of said monoclonal antibody, capable of binding to a deamidated gliadin and a deamidated glutenin and not exhibiting any cross reaction with a non-deamidated gliadin and a non-deamidated glutenin.

3. The antigen-binding molecule as claimed in claim 1, characterized in that it comprises:
(a) at least the variable region of a heavy chain comprising the following three CDRs:
V_{H}-CDR1, comprising a sequence having at least 80% amino acid identity with the sequence SEQ ID NO. 2,
V_{H}-CDR2, comprising a sequence having at least 80% amino acid identity with the sequence SEQ ID NO. 3, and
V_{H}-CDR3, comprising a sequence having at least 80% amino acid identity with the sequence SEQ ID NO. 4; or
(b) at least the variable region of a light chain comprising the following three CDRs:
V_{L}-CDR1, comprising a sequence having at least 80% amino acid identity with the sequence SEQ ID NO. 6,
V_{L}-CDR2, comprising a sequence having at least 80% amino acid identity with the sequence SEQ ID NO. 7, and
V_{L}-CDR13, comprising a sequence having at least 80% amino acid identity with the sequence SEQ ID NO. 8.

4. The antigen-binding molecule as claimed in claim 1, characterized in that it comprises:
(a) at least the variable region of a heavy chain comprising the following three CDRs:
V_{H}-CDR1, comprising the sequence SEQ ID NO. 2,
V_{H}-CDR2, comprising the sequence SEQ ID NO. 3, and
V_{H}-CDR3, comprising the sequence SEQ ID NO. 4, and
(b) at least the variable region of a light chain comprising the following three CDRs:
V_{L}-CDR1, comprising the sequence SEQ ID NO. 6,
V_{L}-CDR2, comprising the sequence SEQ ID NO. 7, and
V_{L}-CDR3, comprising the sequence SEQ ID NO. 8.

5. The antigen-binding molecule as claimed in claim 1, characterized in that it binds to a deamidated gluten protein chosen from a deamidated gliadin and a deamidated glutenin and does not exhibit any cross reaction with a non-deamidated gliadin and a non-deamidated glutenin.

6. The antigen-binding molecule as claimed in claim 1, characterized in that it is produced by the hybridoma deposited according to the Treaty of Budapest at the CNCM on Feb. 25, 2013, under accession number 1-4717, with the Collection Nationale de Cultures de Microorganismes [National Collection of Microorganism Cultures] (CNCM) of the Institut Pasteur of Paris.

7. A complex formed between an antigen-binding molecule as claimed in claim 1 and a deamidated gluten protein.

8. The antigen-binding molecule as claimed in claim 1, for use thereof in a method for detecting the presence of deamidated gluten proteins in a sample.

9. A process for detecting the presence of deamidated gluten proteins in a sample, comprising the following steps:
a) providing a test sample,
b) bringing said sample into contact with an antigen-binding molecule as claimed in claim 1,
c) detecting the complexes possibly formed between said sample and said antigen-binding molecule.

10. A process for detecting the presence of deamidated gluten proteins in a sample, comprising the following steps:
a) providing a test sample,
b) incubating said sample with an antigen-binding molecule as claimed in claim 1, under conditions which allow the formation of complexes between said antigen-binding molecule and the deamidated gluten proteins, in order to obtain an incubation solution,
c) bringing the incubation solution obtained at the end of step b) into contact with a support on which is immobilized a peptide which mimics a deamidated gluten protein, and
d) detecting or quantifying the complexes possibly formed between (i) said peptide which mimics a deamidated gluten protein and which is immobilized on the support and (ii) antigen-binding molecules present in the incubation solution obtained at the end of step b).

11. The process as claimed in claim 9, characterized in that the peptide which mimics a deamidated gluten protein comprises an amino acid sequence chosen from:
(SEQ ID NO. 17)
EPEEPFPQ,
(SEQ ID NO. 18)
EPQEPFPE,
(SEQ ID NO. 19)
EPEQPFPE,
(SEQ ID NO. 20)
QPEEPFPE,
and
(SEQ ID NO. 21)
EPEEPFPE.

12. The process as claimed in claim 10, characterized in that the peptide which mimics a deamidated gluten protein comprises an amino acid sequence LQPEEPFPEQC (SEQ ID NO. 22).

13. A kit for detecting the presence of deamidated gluten proteins in a sample, comprising at least one antigen-binding molecule as claimed in claim 1.


SEQUENCE LISTING (22 sequences)

SEQ ID NO: 1 - protein, 132 aa, Mus musculus
MRVLILLCLFTAFPGILSDVQLQESGPDLVKPSQSLSLTCTVTGYSITSGYSWHWIRQFPGNKLEWMGYIHYSGSTNYNPSLKSRISITRDTSKNQFFLQLNSVTTEDTATYYCASGYFDVWGAGTTVTVSS

SEQ ID NO: 2 - protein, 11 aa, Mus musculus
GYSITSGYSWH

SEQ ID NO: 3 - protein, 16 aa, Mus musculus
YIHYSGSTNYNPSLKS

SEQ ID NO: 4 - protein, 5 aa, Mus musculus
GYFDV

SEQ ID NO: 5 - protein, 131 aa, Mus musculus
MKLPVRLLVLMFWIPASSSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTLVPWTFGGGTKLEIK

SEQ ID NO: 6 - protein, 16 aa, Mus musculus
RSSQSLVHSNGNTYLH

SEQ ID NO: 7 - protein, 7 aa, Mus musculus
KVSNRFS

SEQ ID NO: 8 - protein, 9 aa, Mus musculus
SQSTLVPWT

SEQ ID NO: 9 - DNA, 396 bp, Mus musculus
atgagagtgctgattcttttgtgcctgttcacagcctttcctggtatcctgtctgatgtgcagcttcaggagtcaggacctgacctggtgaaaccttctcagtcactttcactcacctgcactgtcactggctactccatcaccagtggttatagctggcactggatccggcagtttccaggaaacaaactggaatggatgggctacatacactacagtggtagcactaactacaacccatctctcaaaagtcgaatctctatcactcgagacacatccaagaaccagttcttcctgcagttgaattctgtgactactgaggacacagccacatattactgtgcaagtgggtacttcgatgtctggggcgcagggaccacggtcaccgtctcctca

SEQ ID NO: 10 - DNA, 33 bp, Mus musculus
ggctactccatcaccagtggttatagctggcac

SEQ ID NO: 11 - DNA, 48 bp, Mus musculus
tacatacactacagtggtagcactaactacaacccatctctcaaaagt

SEQ ID NO: 12 - DNA, 15 bp, Mus musculus
gggtacttcgatgtc

SEQ ID NO: 13 - DNA, 393 bp, Mus musculus
atgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacttgttccgtggacgttcggtggaggcaccaagctggaaatcaaa

SEQ ID NO: 14 - DNA, 48 bp, Mus musculus
agatctagtcagagccttgtacacagtaatggaaacacctatttacat

SEQ ID NO: 15 - DNA, 21 bp, Mus musculus
aaagtttccaaccgattttct

SEQ ID NO: 16 - DNA, 27 bp, Mus musculus
tctcaaagtacacttgttccgtggacg

SEQ ID NO: 17 - protein, 8 aa, Mus musculus
EPEEPFPQ

SEQ ID NO: 18 - protein, 8 aa, Mus musculus
EPQEPFPE

SEQ ID NO: 19 - protein, 8 aa, Mus musculus
EPEQPFPE

SEQ ID NO: 20 - protein, 8 aa, Mus musculus
QPEEPFPE

SEQ ID NO: 21 - protein, 8 aa, Mus musculus
EPEEPFPE

SEQ ID NO: 22 - protein, 11 aa, Mus musculus
LQPEEPFPEQC